(19) United States
(12) Patent Application Publication
(10) Pub. No.: US 2019/0125700 A1
(43) Pub. Date: May 2, 2019

(54) ANTIBIOTIC COMPOSITIONS COMPRISING METFORMIN AND METHODS OF USING THE COMPOSITIONS

(71) Applicants: Harry Jung, (US); TED & Associates, Georgetown, TX (US)

(72) Inventors: Paul Dabney, Georgetown, TX (US); Harry JUNG, Georgetown, TX (US)

(73) Assignee: TED & Associates, Georgetown, TX (US)

(21) Appl. No.: 15/794,251

(22) Filed: Oct. 26, 2017

(51) Int. Cl. A61K 31/155; A61K 31/496; A61K 31/4745; A61K 31/43; A61K 9/20; A61K 9/28

(52) U.S. Cl. CPC A61K 31/155; A61K 31/496; A61K 31/4745; A61K 31/43; A61K 9/28; A61K 9/2054; A61K 9/2031; A61K 9/2027; A61K 9/2077

(57) ABSTRACT

Compositions comprising 3-(diaminomethylidene)-1,1-dimethylguanidine (metformin) or a pharmaceutically acceptable salt thereof and an antibiotic are provided. The antibiotic can be a β-lactam antibiotic, vancomycin, an aminoglycoside antibiotic, a fluoroquinolone antibiotic, metronidazole, daptomycin or ciprofloxacin. The compositions can be formulated as a controlled-release pharmaceutical tablet having a granular phase which includes metformin and the antibiotic and an extragranular phase which includes a particulate material which provides a diffusion barrier and/or controlled erosion. A method of preventing, treating or inhibiting a bacterial infection is also provided by administering the compositions to a patient in need thereof. The bacterial infection can be an extracellular bacterial infection.

### BACKGROUND

### Technical Field

This application relates generally to antibiotic compositions and methods of using the compositions and, in particular, to antibiotic compositions comprising 3-(diaminomethylidene)-1,1-dimethylguanidine (metformin) and methods of treating bacterial infections therewith.

### BACKGROUND OF THE TECHNOLOGY

Antibiotics, also called antibacterials, are a type of antimicrobial drug used in the treatment and prevention of bacterial infections. They may either kill or inhibit the growth of bacteria.

Antibiotic or antimicrobial resistance is the ability of microbes to resist the effects of drugs. Although some people are at greater risk than others, no one can completely avoid the risk of antibiotic-resistant infections. Infections with resistant organisms are difficult to treat, requiring costly and sometimes toxic alternatives. Bacteria find ways of resisting the antibiotics developed by humans, which is why aggressive action is needed now to keep new resistance from developing and to prevent the resistance that already exists from spreading. Each year in the United States, at least 2 million people become infected with bacteria that are resistant to antibiotics and at least 23,000 people die each year as a direct result of these infections.

Accordingly, there still exists a need for improved antibiotic compositions, particularly antibiotics for the treatment of antibiotic resistant bacterial infections.

### SUMMARY

According to a first embodiment, a composition is provided which can include:

3-(diaminomethylidene)-1,1-dimethylguanidine or a pharmaceutically acceptable salt thereof; and

an antibiotic.

According to a second embodiment, a controlled-release pharmaceutical tablet is provided which can include:

a granular phase comprising 3-(diaminomethylidene)-1,1-dimethylguanidine or a pharmaceutically acceptable salt thereof and an antibiotic; and

an extragranular phase comprising a particulate material, wherein the extragranular phase provides a diffusion barrier and/or controlled erosion;

wherein the granular phase is dispersed in the extragranular phase.

According to a third embodiment, a method of preventing, treating or inhibiting a bacterial infection is provided which includes administering a composition or a controlled-release pharmaceutical tablet as set forth above to a patient in need thereof.

### DETAILED DESCRIPTION

As used herein, where the indefinite article “a” or “an” is used with respect to a statement or description of the presence of a step in a process of this invention, it is to be understood, unless the statement or description explicitly provides to the contrary, that the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus.

The term “invention” or “present invention” as used herein is a non-limiting term and is not intended to refer to any single embodiment of the particular invention but encompasses all possible embodiments as described in the specification and the claims.

As used herein, the term “about” when used to modify a numerical value means a value that is within 10% of that numerical value.

As used herein, the term “wt %” means weight percent.

Also disclosed is a method of preventing, treating or inhibiting bacterial infection. The terms “treat,” “treatment,” and grammatical variants thereof, refer to both therapeutic treatment and prophylactic or preventative measures, wherein the object is to prevent or slow down (lessen) an undesired physiological condition, disorder or disease or obtain beneficial or desired clinical results. Such beneficial or desired clinical results include, but are not limited to, alleviation of symptoms; diminishment of extent of condition, disorder or disease; stabilized (i.e. not worsening) state of condition, disorder or disease; delay or slowing of condition, disorder or disease progression; amelioration of the condition, disorder or disease state, remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

Research has shown that infection causes a stress response in the body by increasing the level of certain hormones such as cortisol and adrenaline. These hormones work against the action of insulin and, as a result, the body's production of glucose increases, which results in high blood glucose levels also known as hyperglycemia. Since bacteria flourish in a glucose rich environment, elevated blood glucose levels may enable bacteria to resist the effects of many antibiotics. Lowering blood glucose levels to a range not considered elevated may therefore create an environment where antibiotic effectiveness is increased.

Hyperglycemia is a common problem encountered in hospitalized patients, especially in critically ill patients and those with diabetes mellitus. Uncontrolled hyperglycemia may be associated with complications such as fluid and electrolyte disturbances and increased infection risk. Studies have demonstrated impairment of host defenses, including decreased polymorphonuclear leukocyte mobilization, chemotaxis, and phagocytic activity related to hyperglycemia. Glucose is a simple sugar that a person acquires from ingesting food. The cells of the body need glucose to obtain energy. The amount of glucose in the blood is controlled by a feedback mechanism involving two hormones, insulin and glucagon. These hormones work to ensure that blood contains the right amount of glucose so that cells can function correctly. High blood glucose levels caused by infections can be serious resulting in reduced effect of antibiotics or no effect of antibiotics impairing the body's attempts to heal. When a patient's blood glucose level is high (elevated), the white blood cells in the body are less able to combat the bad or harmful bacteria. As a result, they accumulate in the body thus causing serious infections. In addition, the white blood cells cannot reach the infection site quickly enough to engulf the bacteria. Experts have long known that high blood glucose levels increase the chances of a dangerous infection in those with diabetes. Infections in the feet and hands of those with diabetes that can't be brought under control with antibiotics can result in amputation. The high glucose levels in blood and tissue allows bacteria to proliferate and contributes to more rapidly developing infections.

Recent clinical data show that the use of drugs to maintain tight blood glucose concentrations decreases morbidity and mortality in critically ill surgical patients. Intensive insulin therapy minimizes derangements in normal host defense mechanisms and modulates release of inflammatory mediators. The principal benefit of intensive blood glucose regulating therapy is a decrease in infection-related complications and mortality. According to the U.S. Centers for Disease Control (CDC) over 29,000,000 Americans suffer from diabetes. Another 86 million people have elevated blood glucose levels. Accordingly, approximately 40% of Americans have blood glucose levels that are elevated and contributing to poor or retarded healing.

The present inventors have discovered that combining a biguanide drug such as metformin, which reduces blood glucose levels, and an antibiotic creates an environment where the administered antibiotic can significantly improve the healing process and healing speed.

Metformin is a member of a class of drugs called biguanides that helps lower blood glucose levels by improving the way the body handles insulin. Biguanides have the following general structure:

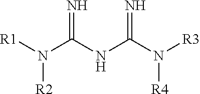

Wherein R1, R2, R3 and R4 are each independently H, an alkyl group or an aryl group. According to some embodiments, a composition comprising a biguanide drug and an antibiotic is provided. According to some embodiments, the biguanide is 3-(diaminomethylidene)-1,1-dimethylguanidine (metformin) wherein R1 and R2 are methyl groups and R3 and R4 are H groups. Metformin is stable by itself under normal storage conditions.

In accordance with an aspect of this invention, a solution providing release or sustained release of metformin and an antibiotic in a tablet or liquid dosage form is provided. The metformin and antibiotic composition as described herein may be formulated for administration by a variety of routes of administration. For example, the metformin and antibiotic may be formulated in a way that is suitable for topical administration; administration in the eye or the ear; rectal or vaginal administration; as nose drops; by inhalation; as an injectable; or for oral administration.

According to some embodiments, the metformin and antibiotic product is formulated in a manner such that it is suitable for oral administration. In accordance with this aspect of the invention, a sustained-release metformin and antibiotic granulation may be distributed in a sustained-release matrix. More particularly, the pharmaceutical tablets in accordance with this aspect of the invention may comprise a granular phase composed of metformin, antibiotics and a hydroxyalkylcellulose. The granular phase may be distributed within an extra-granular phase comprising a particulate material that may provide a sustained-release effect, such as by providing a diffusion barrier and/or controlled erosion. The formed tablet optionally then is provided with a means to obtain a delayed release of active, such as, an enteric coating.

In accordance with a related aspect of the invention, a sustained release metformin and antibiotic pharmaceutical tablet can be prepared by granulating metformin and an antibiotic with a hydroxyalkylcellulose in a wet granulation process. The resulting granulation is dried to an acceptable moisture content, and the dried granulation may optionally be milled and/or screened to achieve a desired granulation particle size. Thereafter, the dried granulation is dry blended with a particulate material capable of forming a sustained-release matrix in which the metformin and antibiotic granules are distributed. The resulting blend is then compressed into a tablet form. Optionally, the tablet can be provided with a means for obtaining delayed release, such as, an enteric coating.

In accordance with another aspect of the invention, there is provided a single tablet dosage form providing metformin and an antibiotic in a sustained-release pharmaceutical tablet in which metformin and an antibiotic granulation are distributed in an extra-granular phase comprising a particulate material capable of providing a sustained-release matrix.

Time-release formulations may be prepared as is known in the art. The compositions described herein may also include pharmaceutically acceptable excipients including, but not limited to, the following: binding agents; fillers; disintegrants; wetting agents; glidants, artificial and natural flavors; sweeteners; artificial or natural colors and dyes; and stabilizers.

According to some embodiments, metformin and an antibiotic are incorporated in a granular phase that is distributed within an extra-granular phase which may together provide a sustained release matrix for the metformin and antibiotic. Alternatively, a non-sustained release form may be used. For the purposes of the instant invention, all pharmacologically active forms of metformin and antibiotics are usable and included in the term “metformin and an antibiotic”. The tablet form optionally is provided with a means for obtaining delayed release of the active, such as, an enteric coating to achieve a further delayed and sustained dissolution profile, as well as a shelf stable pharmaceutical form. Conventional wet granulation techniques may be employed for preparing the metformin and antibiotic granules.

The terms “granule”, “granulation” and “granular phase” refer to particulate agglomerates or aggregates formed by combining the components of the granulation in the presence of a suitable liquid to bind individual particles into aggregated clumps or clusters comprising the individual components of the granulation. Depending on the granulation techniques employed, the selected ingredients, and the desired release properties, the granules, after being dried, can be milled and/or sieved to achieve a desired granule size. Preferably, metformin and an antibiotic are granulated in a manner that maintains the pharmacologic activity of the metformin and an antibiotic. Hence, a suitable liquid used in the wet granulation process is one which is not detrimental to any component of the tablet of interest. Hence, the liquid can be one which contains suitable buffering compounds, for example. A suitable liquid is one which can be adjusted at a basic or acidic pH that enables the formation of the granules while maintaining pharmacologic activity of the metformin and an antibiotic.

According to some embodiments, the metformin and antibiotic composition is provided in the form of a patch, which includes antibiotic dosage forms having different release profiles, as hereinabove described.

The term “therapeutically effective amount” refers to an amount of a pharmaceutically active agent, which when administered to a particular subject, considering the subject's age, weight and other relevant characteristics, will attenuate, ameliorate, or eliminate one or more symptoms of a disease or condition that is treatable with the pharmaceutically active agent.

The amount of metformin and an antibiotic is preferably adjusted to provide conventional therapeutic amounts in the range from about 25 milligrams to greater than 500 milligrams, such as 50, 75, 100, 125, 150, 175, 200, 225, 250, 275, 300, 325, 350, 375, 400, 425, 450, 475, 500, 800 and 1000 milligrams. Larger doses of metformin and an antibiotic, despite formulation size, are contemplated. The amount of hydroxyalkylcellulose needed in the metformin and antibiotic granules to achieve effective sustained release of the metformin and antibiotic in the tablet dosage forms of the invention, and to prevent degradative interactions for tablets containing these two pharmaceutically active compounds, is relatively low. Typically, a suitable and effective amount of hydroxyalkylcellulose in the granular phase is from about 10 to about 30% by weight of the granular phase, with the remaining about 70% to about 90% of the weight of the granular phase being primarily metformin and an antibiotic. The granules can contain other inert excipients as a design choice, such as a lubricant, a glidant and so on. For the sustained-release metformin and antibiotic tablets the relative amount of granular phase to extra-granular phase may vary considerably, depending on the selected tablet dose and the desired release properties. However, the granular phase typically and generally preferably comprises about 30% to about 70% of the combined weight of the granular phase and the extra-granular phase.

The external phase may comprise any particulate material that can be compressed into a tablet form and that provides a sustained-release matrix. Materials having suitable sustained-release properties are generally well known in the art, and typically provide sustained release by providing a diffusion barrier for the active or active ingredients and/or by eroding at a desired controlled rate, with the result being a relatively uniform or constant rate of release of the active ingredient or active ingredients over an extended period of time, such as about 4, about 8, about 16 or about 24 hours. Such sustained release is desirable for maintaining therapeutically effective blood plasma levels of the drug over an extended period of time without requiring re-administration of the drug. Exemplary sustained release materials include, but are not limited to, polyalkylene oxide and a water-swellable or water-erodible polymer selected from the group consisting of polyvinyl pyrrolidone, poly(vinylacetate), copolymers of vinylpyrrolidone and vinylacetate, and blends thereof.

In addition to sustained release, it is desirable to provide metformin and an antibiotic tablet dosage form having delayed release properties. The term “delayed release” as used herein refers to release of the pharmaceutically active compound or compounds that is delayed until after the dosage form has passed through the stomach and into the intestine. Such delayed-release can be achieved by coating the compressed tablet with a polymer coating composition that remains intact in the upper part of the gastrointestinal tract while in contact with acidic gastric fluids, but which readily decomposes or solubilizes at the higher pH in the intestine. An example of such a polymer coating is an enteric coating. An enteric coating generally comprises components soluble in a liquid at a pH 5 or more and includes components that impart resistance to gastric conditions, as known in the art. Exemplary enteric coating materials include anionic acrylic resins, such as methacrylic acid/methyl acrylate copolymer and methacrylic acid/ethyl acrylate copolymer. Polymers of this type include polymers sold by Evonik Nutrition & Care GMBH, Essen, Germany under the tradenames EUDRAGIT® L and EUDRAGIT® S. Other exemplary enteric coating materials include, but are not limited to, hydroxypropylmethylcellulose acetate succinate, hydroxypropylmethylcellulose phthalate, cellulose acetate phthalate, carboxymethylcellulose acetate phthalate and shellac. Mixtures of those compounds also may be used. The enteric coating can comprise from about 1% to about 10% or more of the combined weight of the tablet, depending on the components used in the coating. For example, the enteric coating can comprise about 5%, about 6%, about 7%, about 8%, about 9%, about 10%, about 11%, about 12% or more of the combined weight of the tablet. The enteric coating is selected to provide a desired delayed release of the active to achieve the desired dissolution profile. The composition of the coating and/or the thickness of that coating on the tablet can influence the useful shelf life of the product. Other auxiliary coating aids such as a minor amount of a plasticizer, such as acetyltributylcitrate, triacetin, acetylated monoglyceride, rape oil, olive oil, sesame oil, acetyltriethyl-citrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, polyethyleneglycol, propylene glycol and mixtures thereof in combination with an anti-sticking agent which may be a silicate such as talc, can be used. A flavorant or colorant may be included. The components may be added to the formula in combination with appropriate solvents. The delayed release enteric coating generally can be one which causes a delay of active dissolution, for example, active release from the tablet will not substantially occur until after the dosage form is removed from a simulated gastric fluid in vitro or the stomach in vivo. Essentially, the enteric coating is one which does not dissolve under conditions found in the stomach but dissolves under conditions found in the intestine, for example, based on the acidity of the environment.

Infections are a common cause of patient physician visits and hospital admissions and appropriately identifying the infection and pathogen are desirable for optimal resolution. At times, empiric antimicrobial therapy must be instituted, and knowledge of common pathogens typical for a suspected site of infection, and corresponding effective antimicrobials is necessary while awaiting definitive diagnoses. The goal of metformin and antibiotic therapy is to correctly identify an infection and then select the most appropriate metformin and anti-infective that can sufficiently treat the infection. This, while being as narrow spectrum as possible, providing the best penetration into the infected organ including lowering blood glucose levels, while minimizing adverse reactions, including further organ damage or risks due to antimicrobial therapy itself, such as need for intravenous access, renal toxicity, or acquisition of superinfection such as Clostridium difficile colitis.

Because the kidney and the liver are the primary organs responsible for elimination of drugs from the body, it is important to determine how well they are functioning during antimicrobial administration. In most cases, one is concerned with dose reduction to prevent accumulation and toxicity in patients with reduced renal or hepatic function. In regard to this patent, various antibiotics may be selected, taking into consideration hepatic and renal function, and then coupled with metformin to provide the appropriate antimicrobial response in the most appropriate delivery form.

Along with host factors, the pharmacodynamic properties of antimicrobial agents may also be important in establishing a dosing regimen for metformin and an antibiotic combination drug. Specifically, this relates to the concept of time-dependent vs concentration-dependent killing. Drugs that exhibit time-dependent activity (β-lactams and vancomycin) have relatively slow bactericidal action; therefore, it is important that the serum concentration exceeds the minimum inhibitory concentration (MIC) for the duration of the dosing interval, either via continuous infusion or frequent dosing. Metformin lowers the MIC when combined with time-dependent activity antibiotics. In contrast, drugs that exhibit concentration-dependent killing (aminoglycosides, fluoroquinolones, metronidazole, and daptomycin) have enhanced bactericidal activity as the serum concentration is increased. With these agents, the “peak” serum concentration, and not the frequency of the dosing interval, is more closely associated with efficacy. Metformin lowers the MIC when combined with concentration-dependent activity antibiotics.

To illustrate the impact of this distinction on dosing options, we can take the example of a 70-year-old woman with a creatinine clearance estimated to be 30 mL/min who is being treated with metformin and ciprofloxacin for pyelonephritis caused by E coli. Antimicrobial dosing guidelines suggest that a dose of either 250 mg orally every 12 hours or 500 mg every 24 hours is an acceptable modification for her reduced kidney function. However, given that metformin and ciprofloxacin exhibits concentration-dependent killing, selection of the latter dosing schedule would be more appropriate. In contrast, if the same patient were being treated with metformin and intravenous ampicillin, for which the time above the MIC is more closely related to efficacy, a dose of 1 g every 4 hours would be preferable to 2 g every 8 hours.

According to some embodiments, the antibiotic can be a fluoroquinolone antibiotic having a structure represented by the following formula:

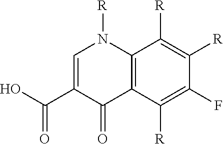

wherein:
each R is, independently, H, a halogen, F, a substituted or unsubstituted alkyl group, a substituted or unsubstituted alicyclic group, a substituted or unsubstituted cycloalkyl group, a substituted or unsubstituted heterocyclic aliphatic amine group, or wherein two or more R groups together may form a cyclic or heterocyclic moiety.

Exemplary antibiotics of the above type include, but are not limited to:

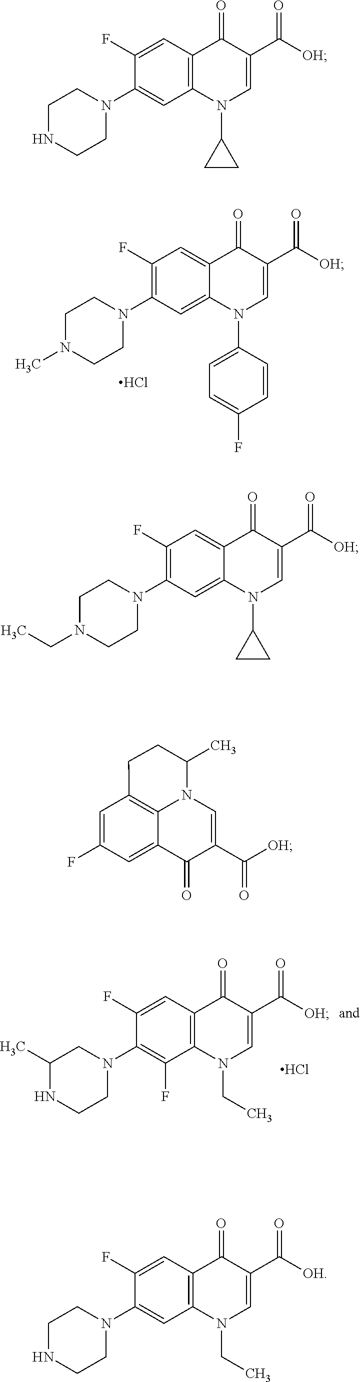

According to some embodiments, the antibiotic is an aminoglycoside. Exemplary aminoglycoside antibiotics include, but are not limited to, streptomycin, kanamycin, daptomycin, tobramycin, gentamicin, vancomycin and neomycin.

According to some embodiments, the antibiotic comprises a β-lactam ring. Exemplary β-lactam antibiotics include, but are not limited to, penams, carbapenams, oxapenams, penems, carbapenems, monobactams, cephems, carbacephems, oxacephems, ampicillin, penicillins and cephalosporins. According to some embodiments, the antibiotic is a β-lactam antibiotic having the following general structure:

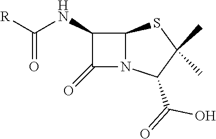

wherein R is a substituted or unsubstituted alkyl group, a substituted or unsubstituted aryl group, or an ether group. Exemplary R groups include benzyl, phenyl, phenoxymethyl 2,6-dimethoxyphenyl and the following groups:

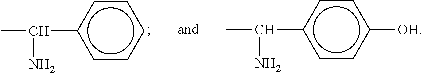

Exemplary antibiotics of the above type include, but are not limited to, Ampicillin, Penicillin G, Penicillin V, Amoxicillin, Oxacillin, Carbenicillin and Methicillin, Nafcillin, Ticarcillin and Piperacillin.

According to some embodiments, the antibiotic is metronidazole.

Other exemplary antibiotics include sulfonamides such as co-trimoxazole (Bactrim) and trimethoprim (Proloprim), and tetracycline antibiotics such as tetracycline (Sumycin, Panmycin) and doxycycline (Vibramycin)

Pathogenic bacteria can be classified as extracellular bacteria or intracellular bacteria on the basis of their invasive properties for eukaryotic cells. Extracellular bacterial pathogens do not invade cells and proliferate instead in the extracellular environment. Extracellular bacteria do not have the capacity to survive the intracellular environment or to induce their own uptake by most host cells. Unlike extracellular pathogens, intracellular pathogens establish themselves within the cell and spend all or part of their life intracellularly.

According to some embodiments, the compositions described herein can comprise metformin and an antibiotic for an extracellular bacteria. According to some embodiments, a method of preventing, treating or inhibiting an extracellular bacterial infection is provided comprising administering a composition comprising metformin and an antibiotic to a patient in need thereof.

Examples of extracellular bacteria include, but are not limited to, Bacillus anthracis, Enterotoxigenic Escherichia coli, Haemophilus influenza, Mycoplasma spp, Pseudomonas aeruginosa, Staphylococcus aureus. Streptococcus pyogenes and Vibrio cholerae.

While the foregoing specification teaches the principles of the present invention, with examples provided for the purpose of illustration, it will be appreciated by one skilled in the art from reading this disclosure that various changes in form and detail can be made without departing from the true scope of the invention.

1. A composition consisting of:
3-(diaminomethylidene)-1,1-dimethylguanidine or a pharmaceutically acceptable salt thereof; and
an antibiotic;
wherein the 3-(diaminomethylidene)-1,1-dimethylguanidine and the antibiotic are both formulated for immediate release;
wherein the 3-(diaminomethylidene)-1,1-dimethylguanidine and the antibiotic are both formulated for sustained release; or
wherein the 3-(diaminomethylidene)-1,1-dimethylguanidine and the antibiotic are both formulated for delayed release.

2. The composition of claim 1, wherein the antibiotic is selected from the group consisting of: a β-lactam antibiotic, vancomycin, an aminoglycoside antibiotic, a fluoroquinolone antibiotic, metronidazole, daptomycin and ciprofloxacin.

3. The composition of claim 1, further comprising a pharmaceutically acceptable carrier.

4. The composition of claim 1, wherein the antibiotic is a fluoroquinolone antibiotic having a structure represented by the following formula: 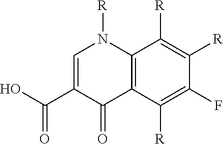
wherein each R is, independently, H, a halogen, F, a substituted or unsubstituted alkyl group, a substituted or unsubstituted alicyclic group, or a substituted or unsubstituted heterocyclic aliphatic amine group, or wherein two or more R groups together form a cyclic or heterocyclic moiety.

5. The composition of claim 1, wherein the antibiotic is a β-lactam antibiotic having the following general structure: 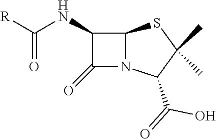
wherein R is a substituted or unsubstituted alkyl group, a substituted or unsubstituted aryl group or an ether group.

6. A controlled-release pharmaceutical tablet comprising:
a granular phase comprising the composition of claim 1; and
an extragranular phase comprising a particulate material, wherein the extragranular phase provides a diffusion barrier and/or controlled erosion;
wherein the granular phase is dispersed in the extragranular phase.

7. The controlled-release pharmaceutical tablet of claim 6, wherein the granular phase further comprises a hydroxyalkylcellulose.

8. The controlled-release pharmaceutical tablet of claim 7, wherein the hydroxyalkylcellulose comprises from about 10% to about 30% by weight of the granular phase.

9. The controlled-release pharmaceutical tablet of claim 6, wherein the granular phase comprises from about 30 to about 70% of the combined weight of the granular and extragranular phases.

10. The controlled-release pharmaceutical tablet of claim 6, wherein the extragranular phase comprises:
polyalkylene oxide; and
a water-swellable or water-erodible polymer selected from the group consisting of polyvinyl pyrrolidone, poly(vinylacetate), copolymers of vinylpyrrolidone and vinylacetate, and blends thereof.

11. The controlled-release pharmaceutical tablet of claim 6, further comprising a coating.

12. The controlled-release pharmaceutical tablet of claim 11, wherein the coating is an enteric coating.

13. The controlled-release pharmaceutical tablet of claim 12, wherein the coating comprises from about 1% to about 12% of the combined weight of the tablet and coating.

14. The controlled-release pharmaceutical tablet of claim 12, wherein the coating comprises a component selected from the group consisting of: an anionic acrylic resin; hydroxypropylmethylcellulose acetate succinate; hydroxypropylmethylcellulose phthalate; cellulose acetate phthalate; carboxymethylcellulose acetate phthalate; shellac and combinations thereof.

15. A method of treating a bacterial infection comprising administering a composition as set forth in claim 1 to a patient in need thereof.

16. The method of claim 15, wherein the bacterial infection is an extracellular bacterial infection.

17. The method of claim 15, wherein the composition is administered to the patient by a method selected from the group consisting of: topical administration;
administration in the eye or the ear; rectal administration; vaginal administration; nasal administration; administration by inhalation; administration by injection; and oral administration.

18. The method of claim 15, wherein the composition is administered to the patient orally in tablet form.

19. A method of treating a bacterial infection comprising administering a controlled-release pharmaceutical tablet as set forth in claim 6 to a patient in need thereof.

20. The method of claim 19, wherein the bacterial infection is an extracellular bacterial infection.
